(19) United States
(12) Patent Application Publication
(10) Pub. No.: US 2010/0049332 A1
(43) Pub. Date: Feb. 25, 2010

(54) LEG PROSTHESIS

(75) Inventors: Einar Kolderup SELVIG, Bergen (NO); Leif VARTDAL, Trondheim (NO)

Correspondence Address: DENNISON, SCHULTZ & MACDONALD, 1727 KING STREET, SUITE 105, ALEXANDRIA, VA, 22314, US

(73) Assignee: INLIEU AS, Trondheim (NO)

(21) Appl. No.: 12/194,099

(22) Filed: Aug. 19, 2008

(51) Int. Cl. A61F 2/74; A61F 2/72

(52) U.S. Cl. 623/27; 623/26

(57) ABSTRACT

Leg prosthesis including a cylinder with a piston and a piston rod, the piston rod being led out of one end of the cylinder and attached to an outer tube of the prosthesis, with the lower part of the cylinder being attached to an inner tube of the prosthesis. The outer tube and the inner tube may be connected to parts of the leg prosthesis, and the cylinder may contain a pneumatic or hydraulic medium. The inner tube and the outer tube provide a cavity for the arrangement of a torsion device, where the outer tube can rotate in both directions in relation to the inner tube and vice versa. The torsion device includes an elastomer fixed to the outer tube, elastomer supports fixed to the outer tube and an elastomer end stop fixed to the inner tube.

### FIELD OF THE INVENTION

The invention relates to a torsion device for a prosthesis, especially a leg prosthesis.

### DESCRIPTION OF RELATED ART

Following amputation of a leg, at the calf, knee, thigh or hip, a patient often receives a prosthesis as a substitute for the lost body part. The stump of the patient is generally connected to the prosthesis by a sleeve, known as a “bit-sleeve”. It is also possible to connect the prosthesis directly to the body part with a titanium screw, so-called osseointegration or tissue-integrated prosthesis. The bit-sleeve is provided with an adaptor and an ankle connected to a foot piece. This kind of leg prosthesis may also comprise a knee and a hip, depending on the amputation level.

Shock absorbers are inserted in the prosthesis, cushioning and attenuating the ground reaction forces, which affect the body during walking, running and jumping. Cushioning and attenuation of this load on the sensitive stump and body can help to reduce stump problems, avoid overload of the muscle and skeletal apparatus, and improve the comfort for the patient. The rotatability of the mechanism compensates for the missing rotatability of the hip joint with thigh amputees due to the rigid stump connection at the pelvis. Opposite to the pelvis, there is a special importance to the interior rotation of the foot. An increased rotational rigidity (racing or initial moment) for small deflections of the shock absorber have been perceived by the patient in field tests as very pleasant and stabilizing.

A torsion mechanism to provide rotatability is disclosed in DE 196 37 173 A1 (U.S. Pat. No. 6,086,615), in which a prosthetic carrier with a compressible volume of fluid enclosed therein is used to support the weight of the patient. A first end portion of the device is fastenable to the leg stump of the patient, and an opposite, second end portion is connectable to a foot prosthesis, and swivels opposite the first end portion around the longitudinal axis of the wearer. First and second end portions are coupled over a torsion bar which resists a rotation in a clockwise direction with a first spring rate and resists a rotation against the clockwise direction with a second spring rate, which is selected to differ from the first spring rate, depending on whether the leg stump of the patient is a right or a left leg stump.

EP1275355 (U.S. Pat. No. 6,827,343) also discloses a torsion device for a prosthesis which consists of a proximal part which can be twisted, in relation to a distal part, in positive and negative rotary directions. The torsion element consists of two separate torsion spring elements possessing differing spring characteristics and formed of two elastomeric ring segments. The segments are each supported on a carrier of the proximal part and on a thrust-piece fixed to the distal part.

EP1276441 discloses a device for height control of a leg prosthesis, comprising a cylinder with a piston and a piston rod led out of the cylinder at one end, the cylinder and the piston rod each being connected with one of two connecting parts, that further may be connected with parts of the leg prosthesis, the cylinder including a pneumatic or hydraulic medium. In connection with the cylinder there is provided a channel for transferring a medium from one side of the piston to the other, through a valve that can be operated by the prosthesis user. The piston is pre-stressed towards an outer end position, particularly by the differential pressure area between the two piston sides.

It is not known in the prior art to provide a prosthesis having a height control which also provides shock absorption, and which is also provided with a torsion device. The known torsion devices are based on several torsion elements, i.e. elastomers, which enable rotation of the prosthesis. By using several elastomers, there are disadvantages of a shorter lifetime and complicated mounting, making it difficult for the user to adapt the prosthesis.

### SUMMARY OF THE INVENTION

It is therefore an object of the invention to improve a torsion device of the described functionality.

It is a further object that the torsion device include only one elastomer.

It is a still further object that the device be easy to adapt and made of simple parts, making the device of the invention less expensive to produce than prior art.

A still further object of the invention is to provide a device in which the torsion device is replaceable or modifiable in a simple manner to adjust the characteristics of the torsion element.

It is further an object of the invention that the height control not affect the torsion device.

Finally, it is an object of the invention to provide a leg prosthesis which is height adjustable and which also provides shock absorption and torsion control.

To achieve these and other objects, the invention is directed to a height adjustable leg prosthesis which preferably includes a cylinder with a piston, the piston having a piston rod, which is led vertically upwards through an upper end wall of the cylinder and fixed to an outer tube. At the lower part, the cylinder is fixed to an inner tube. A suitable gas is filled into the cylinder, on both sides of the piston, and the inner tube is movable in relation to the outer tube to provide height control by means of the extension of the piston and piston rod of the cylinder.

By including such a cylinder and piston in a leg prosthesis, it is possible to perform rapid control and fine adjustment of the prosthesis height, and at the same time, the height control can function as a shock absorber, where this is desirable and practical. Further, it is easy to manually activate, preferably without visible encroachment and it is made of simple parts, preferably elements that are commercially available.

The height control of the prosthesis may be performed without affecting the shock absorbing properties of the prosthesis. Disengagement may be achieved in a simple manner, adjusted to the needs and desires of the prosthesis user.

An important advantage of the invention is that the activation needed to initiate the height control can, if desired, be done without direct hand operation, as a valve can be opened upon a single press with the healthy foot.

It is a favorable feature of the invention to provide a compact embodiment and a safe function.

The leg prosthesis is further provided with means for rotational movement, comprising a torsion device arranged in a cavity provided between the outer tube and the inner tube. In the cavity between the outer and inner tube is arranged an upper bearing at the upper part of the inner tube, and an end cap including a lower bearing and scraper is arranged at the lower part of the inner tube. The upper bearing and the end cap are held in place by the outer tube.

The torsion device preferably includes only one elastomer which is fixed to the outer tube by means of an elastomer mounting. The elastomer is preferably supported by elastomer supports arranged on the outer tube at both sides adjacent to the elastomer, which elastomer supports are preferably limited in movement in the transversal direction by the elastomer support including slits extending in transversal direction, via which slits the elastomer supports are mounted onto pins extending perpendicularly from the outer tube, inwards against the inner tube. The slits have a limited extension to provide the necessary movement for the pre-stressing of the elastomer at the point of mounting. Further, the torsion device includes an elastomer end stop in the form of a vertically fixed element arranged on the inner tube, arranged at the opposite side of the elastomer mounting for the elastomer in the outer tube.

In this way the elastomer will, as the outer tube of the prosthesis rotates due to movement of the user, press against the elastomer supports, which again will abut the elastomer end stop, which results in the elastomer being compressed and an increasing force will be built up in the elastomer, also increasing rotational resistance. As the person using the prosthesis lifts the leg from the ground, the prosthesis will return to its initial position, i.e. the inner tube and the outer tube return to their initial positions due to the force built up in the elastomer. If the inner tube rotates in relation to the outer tube, the elastomer end stop will press on one of the elastomer supports, depending on the rotation direction, and the elastomer support again presses on the elastomer, resulting in the elastomer being compressed and an increasing force building up in the elastomer, and also increasing the resistance to rotation. As the prosthesis is lifted from the ground, the inner tube and the outer tube will return to their initial positions as described above.

In this way, the person using the prosthesis will experience much of the same effects as a person with a normal leg experiences, and in addition has the possibility of adjusting the height of the prosthesis.

The characteristic of the torsion device, especially the elastomer, is preferably adapted to the requirements of the user, such as height, weight, mobility, etc., so as to obtain an optimal prosthesis for the user.

Preferably, the elastomer can be replaced or modified manually in a simple manner so that the patient can adapt the prosthesis to the patient's use. Different elastomers with different characteristics are required for the area of use, e.g. a different characteristics are required if the patient is to run than walk. This effect can be obtained by using elastomers with different characteristics.

If a person requires different characteristics in the different rotation directions, the elastomer can be provided with one or more cutouts resulting in the elastomer exhibiting different flexibility in different directions.

Other ways for altering the characteristics of the elastomer are, for example, to provide the elastomer with one or more tapering ends or to provide an elastomer support especially adapted to receive the elastomer in a favorable way.

Further advantageous features will appear from the following example.

### BRIEF DESCRIPTION OF THE DRAWINGS

The invention will be described hereinafter with reference to the accompanying drawings, wherein:

FIG. 1 shows a side, cross-sectional view of an embodiment of the invention;

FIGS. 2a-d shows details of a torsion device according to the invention in perspective view; and

FIGS. 3a and 3b show different embodiments of the elastomer of the torsion device in perspective view.

### DESCRIPTION OF THE PREFERRED EMBODIMENTS

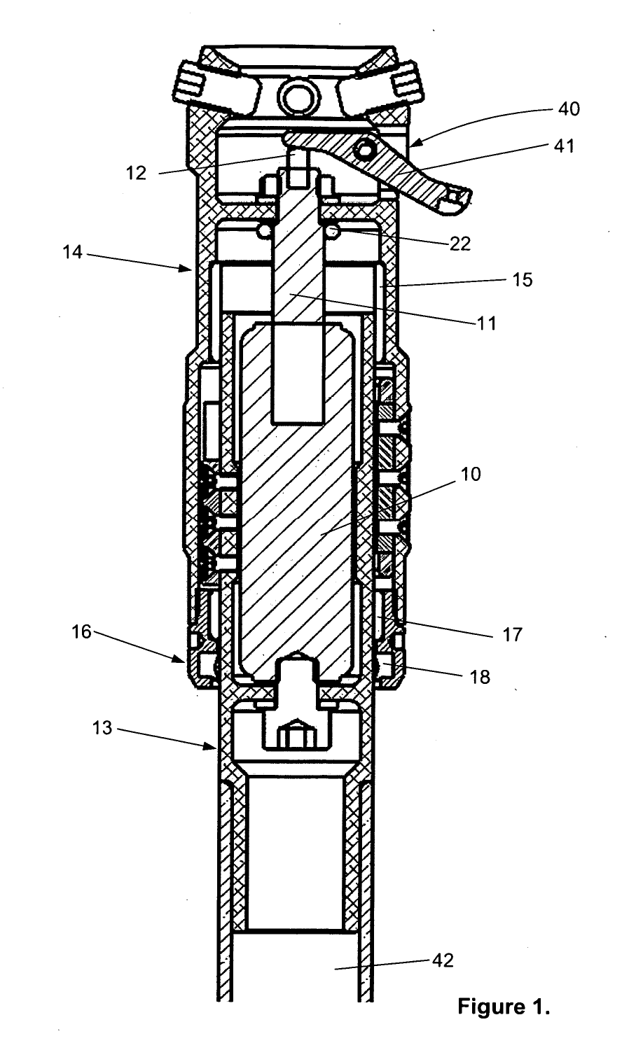

The device according to FIG. 1 comprises a cylinder 10 with a piston (not shown), which piston has a piston rod 11, which is led vertically upwards through the upper end wall of the cylinder 10. A suitable gas, e.g. nitrogen, is filled into the cylinder 10, on both sides of the piston.

To make the neutral or rest-position of the piston adjustable, a valve (not shown) is provided, with connections to both sides of the piston. The valve is normally closed, but can be opened via spring tension with an activation lever 12, which is led through the piston rod 11.

The lower end of the cylinder 10 is fixed to an inner tube 13, which inner tube 13 is adapted for this at its lower part.

Correspondingly, the piston rod 11 is fixed to an outer tube 14.

The inner telescopic tube 13 overlaps in all possible positions of the gas spring with the outer telescopic tube 14. The telescopic tubes 13 and 14 may be made of plastic, especially plastic reinforced with carbon fibers or a light metal, particularly titanium.

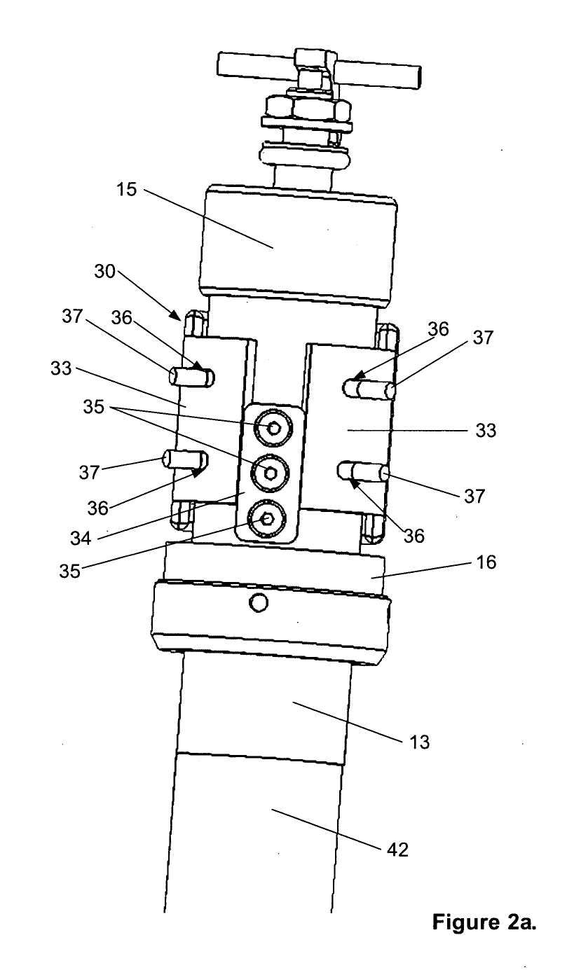

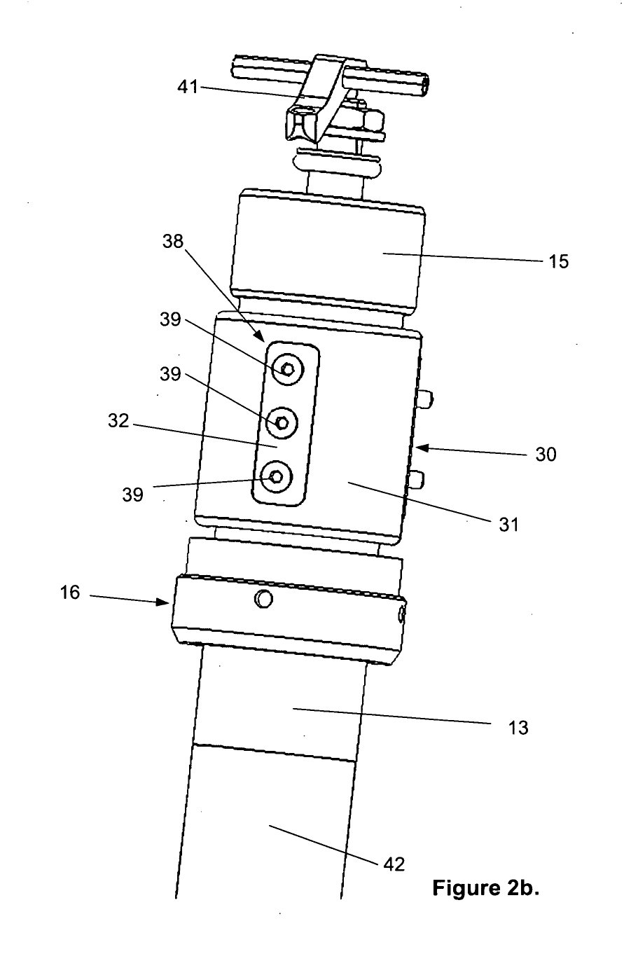

Referring now also to FIGS. 2a-d, in addition to FIG. 1, FIGS. 2a and 2b show a prosthesis without the outer tube 14 to reveal details of a torsion device 30 according to the invention. FIGS. 2c and 2d show details of the torsion device 30 and the outer tube 14. At the upper part of the inner tube 13, there is arranged an upper bearing 15, which is adapted to the outer circumference of the inner tube 13. At the lower part of the inner tube 13, there is arranged an end cap 16, which is adapted to the outer circumference of the inner tube 13 and which holds a lower bearing 17 and scraper 18 which surrounds the inner tube 13. The upper bearing 15 and the end cap 16 are adapted to be arranged and fixed by the outer tube 14. The upper bearing 15 and the end cap 16 provide a cavity between the inner tube 13 and the outer tube 14, which is limited by the upper bearing 15 and the lower bearing 17 of the end cap 16 in the vertical direction, into which a torsion device 30 according to the invention can be arranged. The outer tube 14 is in this way fixed to the end cap 16 and the piston rod 11. Preferably, an end stop damper 19, such as an O-ring, is arranged between the piston rod 11 and the outer tube 14 to provide damping between the inner piston rod 11 and the outer tube 14.

The torsion device 30 includes an elastomer 31, an elastomer mounting 32, two elastomer supports 33 and an elastomer end stop 34. The elastomer end stop 34 is arranged on the inner tube 13 by suitable fastening means such as threaded bolts 35 in threaded holes (not shown) provided in the inner tube 13. Preferably, the elastomer end stop 34 has a substantially rectangular shape extending in the vertical direction of the inner tube 13. The elastomer end stop 34 is preferably arranged so that when the prosthesis is compressed, the elastomer end stop 34 nearly abuts the upper bearing 15 and when the prosthesis is extended, the elastomer end stop 34 nearly abuts the lower bearing 17 of the end cap 16.

At each side of the elastomer end stop 34 is arranged an elastomer support 33, which preferably has a substantially rectangular shape, extending in the vertical direction of the inner tube 13, which is provided with parallel slits 36 having limited extension in the transversal direction of the elastomer supports 33. The elastomer supports 33 are, via the slits 36, arranged movably in the transversal direction to the outer tube 14 by means of protruding pins 37 provided in the outer tube 14, extending inward against the inner tube 13 and into the slits 36. The elastomer supports 33 extend a distance in the transversal direction of the inner tube 13. At the opposite side of the inner tube 13 where the elastomer end stop 34 is arranged, an elastomer 31 is arranged, which preferably has a substantially rectangular shape extending in the circumferential direction of the outer tube 14 and inner tube 13, and which is provided with a centrally arranged opening 38 (FIG. 2b) via which the elastomer 31 is arranged on the outer tube 14 by means of an elastomer mounting 32. The elastomer mounting 32 is adapted to the opening 38 of the elastomer 31 or vice versa. Preferably the opening 38 is substantially rectangular and extends in the vertical direction of the elastomer 31 and the elastomer mounting 32 is preferably rectangular and extends in vertical direction of the outer tube 14. The elastomer mounting 32 is somewhat larger than the opening 38 and is provided with tapering ends to provide an easy mounting and to fix the elastomer 31 securely to the outer tube 14. The elastomer mounting 32 is fixed to the outer tube 14 by means of, for example, threaded bolts 39 which engage with threaded holes provided in the outer tube 14. The shape of the elastomer mounting 32 also facilitates the pre-stressing of the elastomer 31. In this way, the elastomer supports 33 are arranged adjacent to the elastomer end stop 34 and the elastomer 31 extends between the elastomer supports 33 and in this way the ends of the elastomer 31 abut the elastomer supports 33. The elastomer 31 preferably has a length such that it at the time of mounting it is pre-stressed between the elastomer supports 33 in such a way that the elastomer supports 33 abut the elastomer end stop 34. This is to prevent slack in the torsion device 30 to improve the comfort for the person using the prosthesis.

In this way, the elastomer 31, elastomer mounting 32, elastomer supports 33 and elastomer end stop 34 provide a torsion device 30 according to the invention.

The upper outer telescopic tube 14 preferably further includes an axial slit 40 (FIG. 1) arranged at the upper end, for receiving a release lever 41 connected to the activation lever 12. The release lever 41 can be arranged to open the valve upon either downwards or upwards pressure. In the illustrated examples it is made as a double lever, so that it opens the valve upon an upward pull on a string (not shown).

It should be mentioned that the lower end of the inner tube 13 can be joined to a foot piece or an extension tube 42 which is joined to a foot piece. Only the extension tube 42 is shown in the drawings. Either the inner tube 13 or the extension tube 42 or both may be provided with fastening means to mount them securely together.

Correspondingly, the outer tube 14 is at its upper end provided with means for attachment to a leg part of the prosthesis.

In this way, there is provided a leg prosthesis having an adjustable length and which provides shock absorption and which has a torsion device 30 for facilitating rotational movement. Upon walking normally with the leg prosthesis, the compressible medium, the gas inside the cylinder, will function as a shock absorber. The pressure of the shock absorbing gas can be varied, so that the shock absorbing characteristic can be adjusted according to need, depending on the weight of the person etc.

The piston with valve can be opened by placing pressure on the activation lever 12, which protrudes through the piston rod 11. Upon opening of the valve, the pressure medium can flow through a channel (not shown), which communicates with both sides of the piston.

In order to lengthen the leg prosthesis, the user opens the valve, and shifts weight to the healthy foot, so that the leg prosthesis is detached. The area difference between the two sides of the piston, the pressure in the cylinder 11, the area of the channel, and the extent of an eventual additional pre-stressing, will control the speed of the expansion. This speed can thereby be determined accordingly, by choosing suitable dimensions for these elements.

To shorten the leg prosthesis, the user must, with the valve open, load the leg prosthesis with weight, until the piston is pushed into the cylinder 10 to the desired degree. Then the valve is closed.

The use of the torsion device 30 will now be described. In use, i.e. as a person is walking, running or engaging in other activities, the upper part of the prosthesis, i.e. the outer tube 14 and the inner tube 13, is exposed to forces which result in rotation of the outer tube 14 or inner 13 tube in relation to one another due to movements by the person. For example, the inner tube 13, extension tube 42 and the foot piece (not shown) will not move after the foot piece has made contact with the ground. Rotational movement will thus rotate the outer tube 14 and the elastomer 31, fixed to the outer tube 14, and rotates and pushes one of the elastomer supports 33, depending on the direction of rotation, the elastomer support 33 sliding in the tangential direction on the pins 37 and coming into contact with the fixed elastomer end stop 34. The elastomer 31 will be compressed at the one side that abuts the elastomer support 33 in question and a force is built up in the elastomer 31 which will return the outer tube 14 to its initial position in relation to the inner tube 13, extension tube 14 and foot piece (not shown) as the person lifts the prosthesis or the forces acting on the prosthesis are changed in direction due to movement of the person. If the lower part of the prosthesis, i.e. the inner tube 13, extension tube 42 and the foot piece (not shown) rotates in relation to the outer tube 14, the elastomer end stop 34 on the inner tube 13 will press on the one side that abuts the elastomer support 33 in question, depending on the direction of rotation, and a force is built up in the elastomer 31 which will return the inner tube 13, and thus also the extension tube 42 and foot piece (not shown) attached to it, to its initial position in relation to the outer tube 14 as the person lifts the prosthesis or the forces acting on the prosthesis are changed in direction due to movement of the person.

In this way, the torsion device 30 also provides resistance as the rotation angle increases and the person will experience normal rotational movements of the leg.

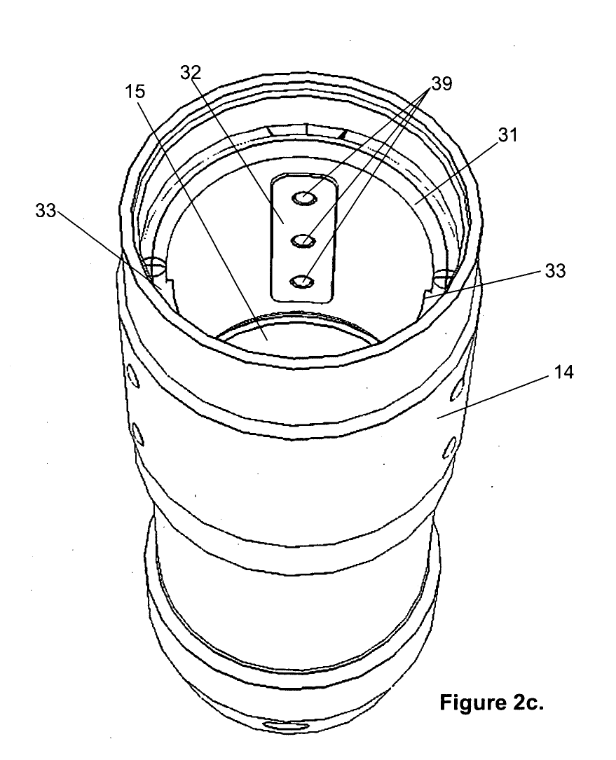

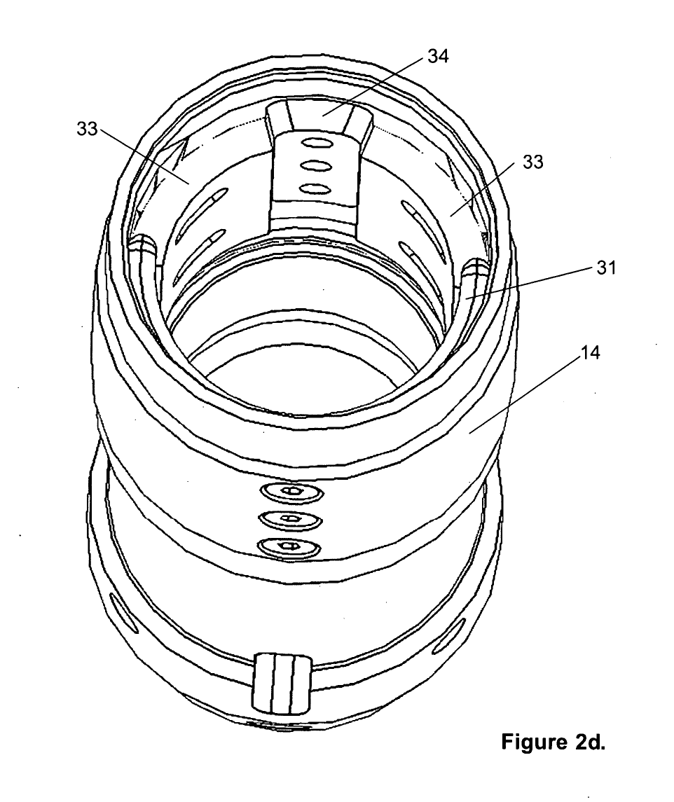

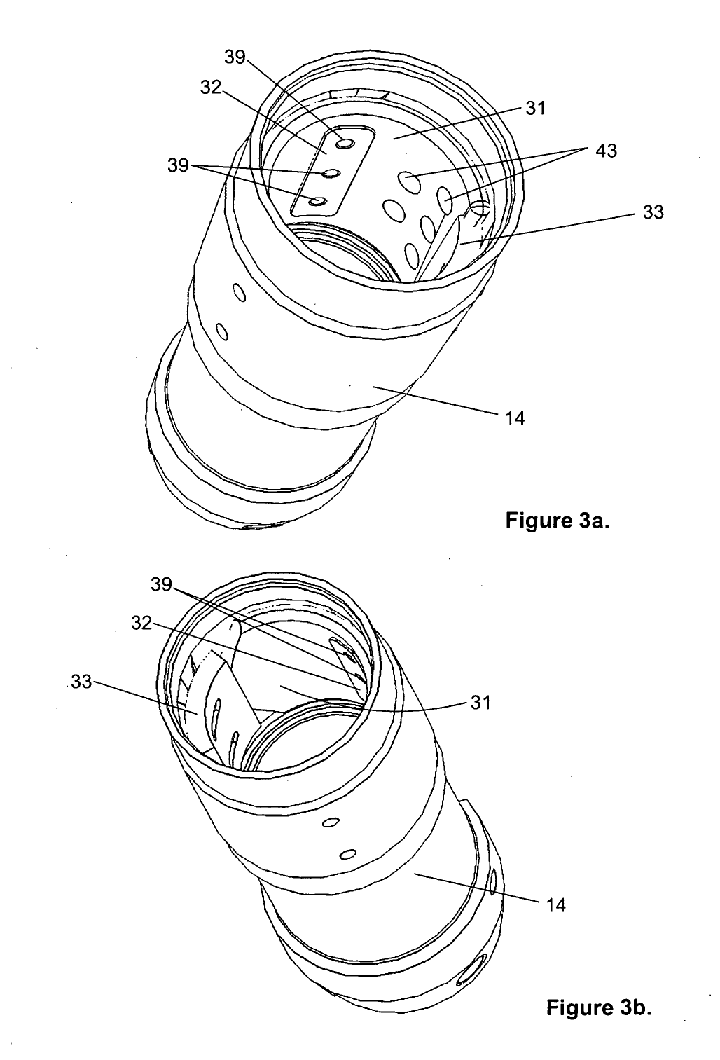

The elastomer 31 is adapted to the person using the prosthesis as are other parts of the prosthesis. The elastomer 31 can thus have different characteristics and preferably it is easy to interchange or modify the elastomer 31 manually so that the user can choose different elastomers or modify the elastomer depending on the activity the user is to perform. For example, an elastomer with lower flexibility will be used during running and walking in the mountains where the torsion device 30 is exerted to greater forces than for normal walking on a street. The flexibility characteristics of the elastomer 31 is are preferably altered by using different materials, but the elastomer can also be adapted of modified by, for example, providing the elastomer with cutouts 43, as shown in FIG. 3a, making it more flexible. This can also be done if the user needs different characteristics for rotation in one direction in relation to another. By providing the elastomer 31 with one or more cutouts 43 at one end of the elastomer, the elastomer will be more flexible in this direction of rotation than the other which is not provided with such cutouts 43 and thus different characteristics are obtained for the elastomer in different directions of rotation.

The characteristics of the elastomer 31 can also be adapted if one or both ends of the elastomer facing the elastomer supports 33 have different shapes so that the flexibility decreases as the elastomer is pressed together and the resistance increases the more the elastomer is pressed together. An example of this is shown in FIG. 3b where one end facing an elastomer support 33 is provided with a tapering end changing the characteristics of the elastomer 31.

The characteristics of the torsion device 30 can also be adapted if the elastomer supports 33 exhibit different shapes on the side adjacent to the elastomer 31 so that the elastomer 31 experiences greater resistance in pressing on the elastomer support 33. An example of this would be if the elastomer support 33 at the end facing the elastomer 31 exhibits a conical shape while the elastomer 31 at the end facing the elastomer support 33 exhibits a recess adapted the conical part of the elastomer support 33 in such a way that the elastomer 31 experiences greater resistance as it is pressed against the elastomer support 33.

The torsion device 30 can thus be provided with different characteristics in many different ways and the embodiments described are only are examples and not to be considered as limiting for the scope of the invention.

The design of the elastomer 31 as regards different characteristics must be adapted to the person using the prosthesis, i.e. such as height, weight, mobility, activity and so on, which is not evident for one of ordinary skill.

The great advantage in that all the components being substantially rectangular is that tearing of the components of the device is low and the device will have a long lifetime.

A number of modifications of the invention are contemplated.

The outer tube can have different shape at the upper end to fit different connecting prosthetic parts.

The inner tube can have different shape at the lower end to fit different connecting prosthetic parts.

The elastomer can be made of different materials and have different shapes.

The elastomer mounting can have different shape for different characteristics, for example to pre-stress the elastomer at the time of mounting.

The elastomer supports can have different shapes to facilitate the effect of the torsion device, for example, the elastomer supports can be conical at one side and are adapted to a recess in the elastomer to provide increasing resistance as the elastomer pushes against the elastomer supports or vice versa.

The gas spring may be mounted in the opposite direction, so that the cylinder is connected to the outer tube and the piston rod is connected to the inner tube.

The piston may be provided with a screw spring or gas spring on the opposite side, increasing the expansion.

The gas spring may comprise an extra piston in the inner of the cylinder, enclosing a gas pillow. This is particularly pertinent when the area of the piston rod is too small to provide a sufficient pressure difference.

The activation lever for height control can be connected to an electrical relay or a remote control, which can receive power from a battery, carried on the body of the user.

The device for height control can be provided with different forms of controlled regulation, either stepless or in fixed steps. This may also be done by means of a service device on a control panel, which can be provided with different programs for adjusting the prosthesis level. By programming, it is possible to adjust the prosthesis to different ranges of application, for example different walking paths. Such a program can be coordinated with regulation of the shock absorbency, so that it is adjustable for the current terrain.

It is also possible to adjust the level regulation for height control so that it is triggered by a pressure sensor.

The cylinder can be provided with an incompressible medium, for example hydraulic oil or gas under high pressure, and to provide the shock absorption by means of a separate compressible piece of material, or a separate screw spring or gas pillow connected to the cylinder.

What is claimed is:

1. A leg prosthesis comprising a cylinder (10) with a piston and a piston rod (11), said piston rod (11) being led out of one end of the cylinder (10) and attached to a outer tube (14) of the prosthesis, and the lower part of the cylinder (10) attached to an inner tube (13) of the prosthesis, the outer tube (14) and the inner tube (13) may again be connected to parts of the leg prosthesis, and the cylinder (10) containing a pneumatic or hydraulic medium, and which inner tube (13) and outer tube (14) provides a cavity for the arrangement of a torsion device (30), which outer tube (14) can rotate both ways in relation to the inner tube (13) and vice versa,
the torsion device (30) including an elastomer (31) fixed to the outer tube (14), elastomer supports (33) fixed to the outer tube (14) and an elastomer end stop (34) fixed to the inner tube (13).

2. Leg prosthesis according to claim 1, wherein the elastomer supports (33) are provided with one or more slits (36), extending in the transversal direction of the elastomer supports (33), which slits (36) have a limited extension in the transversal direction.

3. Leg prosthesis according to claim 1, wherein the elastomer supports (33) are fixed movably to the outer tube (14) by means of pins (37) arranged in the outer tube (14) extending perpendicularly inwards against the inner tube (13) engaging the slits (36) of the elastomer supports (33).

4. Leg prosthesis according to claim 1, wherein the elastomer (31) is provided with a centrally arranged opening (38), preferably with a rectangular shape and preferably extending in the vertical direction of the elastomer (31), via which opening (38) the elastomer (31) is fixed to the outer tube (14) by means of an elastomer mounting (32).

5. Leg prosthesis according to claim 4, wherein the elastomer mounting (32) is adapted to the opening (38) of the elastomer (31) and preferably is provided with tapering ends to provide easy mounting and pre-stressing of the elastomer (31).

6. Leg prosthesis according to claim 1, wherein the cavity between the inner (13) and outer tube (14) is provided by means of an upper bearing (15) and an end cap (16) adapted the circumference of the inner tube (13).

7. Leg prosthesis according to claim 6, wherein the end cap (16) holds a lower bearing (17) and a scraper (18).

8. Leg prosthesis according to claims 1, wherein the outer tube (14) is constructed and arranged to be fixed to the end cap (16) and the piston rod (11).

9. Leg prosthesis according to claim 1, wherein the elastomer (31) is provided with one or more cutouts (43) to provide different characteristic for the elastomer (31).

10. Leg prosthesis according to claim 1, wherein the elastomer (31) is provided with one or more tapering ends to provide different characteristics for the elastomer (31).

11. Leg prosthesis according to claim 1, wherein the elastomer supports (33) exhibit different shapes on the side adjacent to the elastomer (31) to provide different characteristic for the torsion device (30).

12. Leg prosthesis according to claim 11, wherein the elastomer (31) is adapted in shape to the shape of the elastomer supports (33).

13. Leg prosthesis according to claim 1, wherein the elastomer (31) and the elastomer supports (33) are interchangeable.

14. Leg prosthesis according to claim 1, wherein the piston rod (11) being led out of one end of the cylinder (10) is attached to the inner tube (13) of the prosthesis, and the lower part of the cylinder (10) is attached to the outer tube (14) of the prosthesis.
